(19) United States
(12) Patent Application Publication
(10) Pub. No.: US 2019/0231933 A1
(43) Pub. Date: Aug. 1, 2019

(54) HUMAN MESENCHYMAL STROMAL CELL SHEET ENHANCES ALLOGRAFT REPAIR IN A MOUSE MODEL

(71) Applicant: Board of Supervisors of Louisiana State University and Agricultural and Mechanical College, Baton Rouge, LA (US)

(72) Inventor: Yufeng DONG, Shreveport, LA (US)

(21) Appl. No.: 16/264,717

(22) Filed: Feb. 1, 2019

Related U.S. Application Data
(60) Provisional application No. 62/625,285, filed on Feb. 1, 2018.

(51) Int. Cl. A61L 27/38; A61L 27/54; A61L 27/58; A61L 27/28; A61L 27/36; A61L 27/14; A61L 27/04

(52) U.S. Cl. CPC A61L 27/3834; A61L 27/54; A61L 27/58; A61L 27/28; A61L 27/3847; A61L 2300/412; A61L 27/14; A61L 27/04; A61L 2430/02; A61L 2420/02; A61L 27/365

(57) ABSTRACT

A tissue engineered periosteum for a patient comprising a sheet of stem cells. A method of repairing bone injury on a patient comprising implanting into the patient an engineered bone graph comprising a scaffold wrapped in a tissue engineered periosteum. A tissue engineered bone graph comprising a scaffold and a tissue engineered periosteum wrapped around an exterior of the scaffold.

### CROSS REFERENCE TO RELATED APPLICATIONS/PRIORITY

The present invention claims priority to U.S. Provisional Patent Application No. 62/625,285 filed Feb. 1, 2018, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

Limb salvage procedures following massive segmental bone loss due to traumatic extremity injuries or skeletal tumor resections are a major challenge in the field of orthopedics. Large bone defect surgeries like these require devitalized segmental allograft transplantations to replace missing host bone segments; however, significant problems often arise due to the impaired ability of the devitalized allograft to incorporate into the host bone since lacking functional bone forming cells inside allograft.

### SUMMARY

Wherefore, it is an object of the present invention to overcome the above mentioned shortcomings and drawbacks associated with the current technology.

The presently claimed invention relates to methods and devices including a tissue engineered periosteum for a patient comprising a sheet of stem cells. According to a further embodiment the stem cells are mesenchymal stromal cells (MSC). According to a further embodiment the MSC are isolated from a bone marrow of the patient. According to a further embodiment the passage number for the MSC is 10, 9, 8, 7, 6, 5, 4, 3, 2 or 1. According to a further embodiment the passage number for the MSC is 3 or less. According to a further embodiment the passage number for the MSC is 3.

The presently claimed invention further relates to devices and methods of repairing bone injury on a patient comprising implanting into the patient an engineered bone graph comprising a scaffold wrapped in a tissue engineered periosteum. According to a further embodiment the tissue engineered periosteum includes a sheet of stem cells. According to a further embodiment the tissue engineered periosteum includes a sheet mesenchymal stromal cells (MSC). According to a further embodiment the scaffold is formed of one or more of an allograph, an autograph, and/or of one of one or more metals, one or more synthetic polymers, and a combination of one or more metals and one or more synthetic polymers. According to a further embodiment the bone injury is one of a traumatic extremity injury, a skeletal tumor resection, and a large bone defect.

The presently claimed invention is further related to methods and tissue engineered bone graphs comprising a scaffold and a tissue engineered periosteum wrapped around an exterior of the scaffold. According to a further embodiment the scaffold is an allograph. According to a further embodiment wherein the scaffold is an autograph. According to a further embodiment the scaffold is formed of one of one or more metals, one or more synthetic polymers, and a combination of one or more metals and one or more synthetic polymers. According to a further embodiment the tissue engineered periosteum is a sheet of low passage mesenchymal stromal cells (MSC). According to a further embodiment wherein the passage number for the MSC is three or two. According to a further embodiment the tissue engineered periosteum is wrapped around an area adjacent to a terminal end of the scaffold.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIGS. 1A-1D show long-term cell culture induces aging in MSCs. FIG. 1A: Representative Flow Cytometry histograms showing CD73, CD105 and CD90 positive MSCs in cultured P3 and P10 MSCs. FIG. 1B: Quantified percentage of CD73, CD105 and CD90 positive MSCs in P3 and P10 MSCs from 6 patients. FIG. 1C: Representative growth curve of P3 and P10 MSCs from one donor. FIG. 1D: Cumulative population doubling time (DT) of P3 and P10 MSCs is shown in hours (Hr). *P<0.05.

FIGS. 2A-2B show cell morphology change during culture and generation of MSC sheets. FIG. 2A: P3 and P10 MSCs were seeded at different densities for 24 hours and visualized by Giemsa staining. Scale bar, 100 um. FIG. 2B: Expression of ECM related genes was tested by Real-time PCR using total RNA isolated from P3 and P10 MSC sheets, including the type 1 collagen (Cola1), fibronectin, and integrinβ1. *P<0.05.

FIGS. 3A-3B show gross observations before and after mouse allograft surgery. FIG. 3A: Respiration rates were measured by visually counting the number of chest movements per minute (breaths/min) (n=6) at day 0 (D0), day 1(D1), day 2(D2), and day 3(D3). FIG. 3B: Values represent the weight change compared with the starting weight (D0) before surgery and after surgery at week 1 (W1), week 2 (W2), week 3 (W3), week 4 (W4), week 5 (W5), week 6 (W6).

FIGS. 4A-4C show enhanced callus formation in both MSC sheets wrapped allografts at 3 and 6 weeks post-surgery. FIG. 4A: Representative X-ray images of allograft healing at 3 and 6 weeks post-surgery. FIG. 4B: Representative Micro-CT images (cross-section) of allograft healing at 3 and 6 weeks post-surgery. FIG. 4C: Quantification of the bone volume (BV) over total tissue volume (TV) shows a greater bony callus formation in the P3 MSC sheets groups in comparison to the P10 MSC sheets groups (n=6). *P<0.05.

FIGS. 5A-5B show enhanced biomechanical properties in MSC sheet-wrapped allografts at 6 weeks post-surgery. Mechanical properties of femur samples (n=6) in groups of allograft alone, P3 MSC sheet/allograft, and P10 MSC sheet/allograft. FIG. 5A: Torsional rigidity. FIG. 5B: Maximum torque. *P<0.05.

FIGS. 6A-6C show enhanced chondrogenesis in aged MSC sheet wrapped allografts. FIG. 6A: Representative images of callus between host bone and allograft in 3 groups. Histomorphometrical analysis of Alcian blue/Hema/Orange G (AB/H/OG) stained sections at 3 weeks (left) and 6 weeks (right) post-surgery. Specific regions are labeled as follows: b, bone; f, fibrous tissue; c, cartilage tissue. FIG. 6B: Quantification of percentage of the bone, fibrous tissue, cartilage tissue in total callus at 3 weeks (n=6). FIG. 6C: Quantification of percentage of the bone, fibrous tissue, cartilage tissue in total callus at 6 weeks (n=6). Scale bar, 400 um. *P<0.05.

FIGS. 7A-7B show enhanced osteoclast activity and callus remodeling in young P3 MSC sheet wrapped allografts. FIG. 7A: Representative images of TRAP staining in new callus at 3 weeks (left) and 6 weeks (right) post-surgery with high magnification. FIG. 7B: Quantification of the percent ratio of TRAP staining positive osteoclast area vs total callus area (n=6) at 6 weeks post-surgery. Allograft is labeled as: a. Scale bar, 400 um. *P<0.05.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

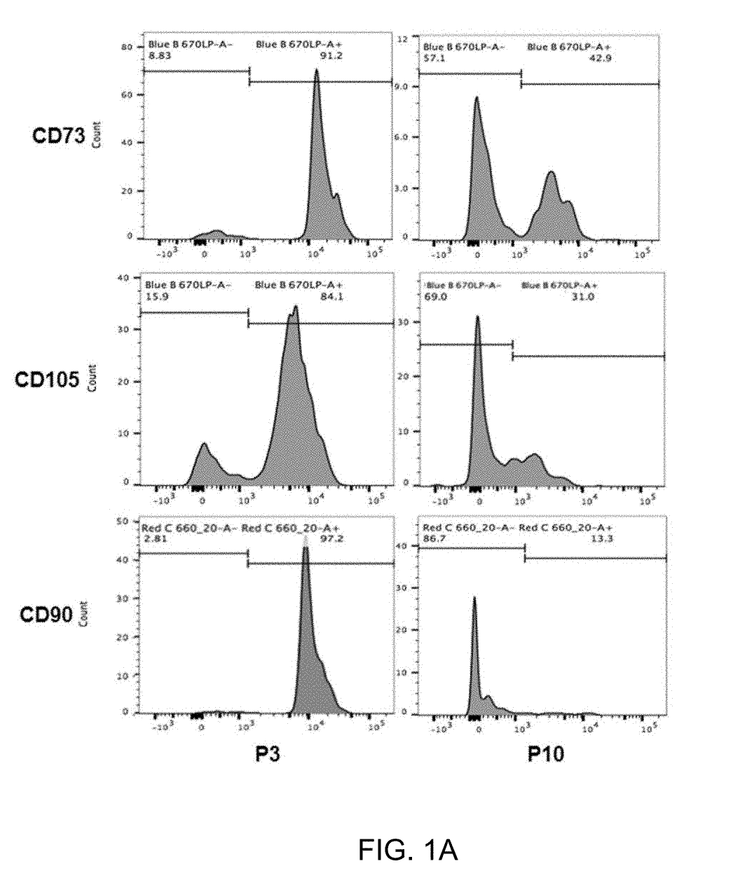

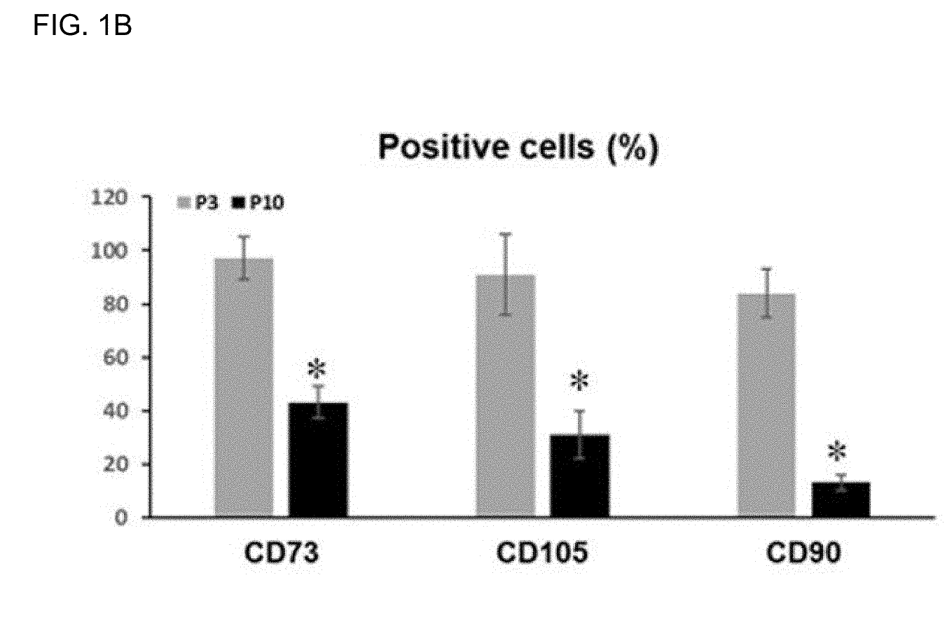

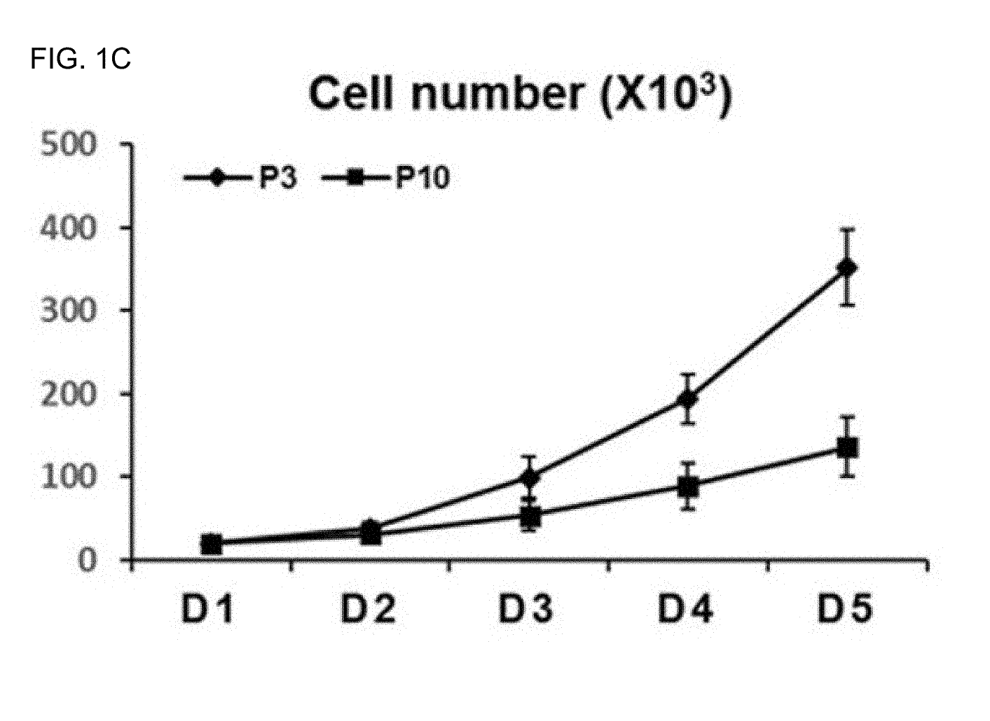

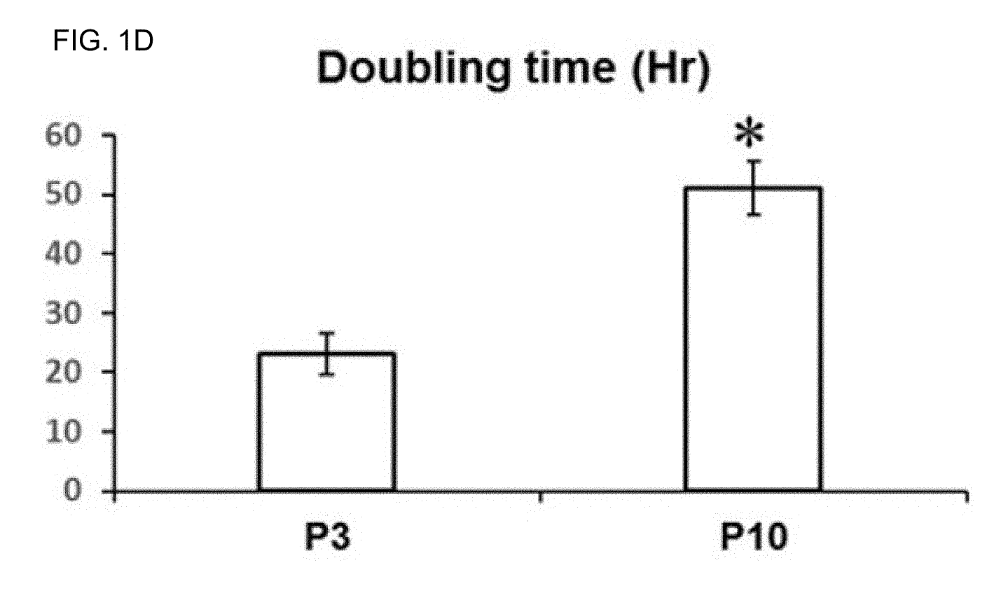

Turning now to FIGS. 1A-7B, a brief description concerning the various components of the present invention will now be briefly discussed.

To determine whether cell sheets generated with long-term passaged (P10) aging human mesenchymal stromal cells (MSCs) could be used for bone tissue regeneration as tissue engineered periosteum in a femoral allograft mouse model similar to fresh passaged (P3) young MSCs. At 3 weeks after transplantation of MSC sheets, results showed more bony callus formed between allograft and host bone ends in both young P3 MSC and aged P10 MSC sheet-wrapped groups when compared to allograft alone. At 6 weeks, while both MSC sheet-wrapped allografts showed more bony callus formation when compared to allograft alone groups, the bony callus size in aged P10 MSC sheet groups was significantly less than young P3 MSC sheet groups. Biomechanical testing confirmed that P3 MSC sheet-grafted femurs had the highest biomechanical strength in the three groups. Histology sections showed that the area of the chondriod callus in the aged P10 MSC sheet groups was significantly larger than in P3 MSC sheet groups. Finally, a significant increase of chondro-osteoclast activity was observed in the P3 MSC sheet-grafted femur. Our data demonstrates that extensive long-term culture-induced MSC aging impaired their osteogenic ability and subsequent bony callus formation, and could be used to induce cartilaginous callus formation.

### INTRODUCTION

One potential treatment strategy entails isolating mesenchymal stromal cells (MSCs) from the patient, expanding them in culture to form a cell sheet, and wrapping cell sheets on devitalized allografts as a tissue-engineered periosteum prior to transplantation. Following transplantation, the MSCs are exposed to endogenous factors within the injured and healing region that promote their osteogenic differentiation, resulting in increased bone callus formation and enhanced osteointegration of the allograft and the adjacent patient bone. Due to the low frequency (0.01% to 0.001%) of MSC in total bone marrow cells, it is essential to culture and populate MSCs in vitro before putting them to therapeutic use. However, in vitro culture has proven to be difficult since the telomere length shortens after each division cycle, leading to a gradual cell aging with an increased cellular senescence and a decreased culture life span. Thus, it is necessary to evaluate the regenerative capacity of long-term ex vivo expanded aging MSC for tissue regeneration.

The inventor has demonstrated the therapeutic effect of early passaged young (P3) primary mouse MSCs following short-term cell sheet culture by maintaining their stromal cell characteristics (Oct4, Sox2, Nanog, and CD105 expression). Furthermore, the inventor has identified the optimal cell number for generating appropriately sized cell sheets in 24 hours using mouse young MSCs. The inventor's short-term cultured MSC sheets showed a significant increase of bone callus formation around allografts. Additionally, prolonged MSC culture provides extra time for the surgeon to have a flexible transplantation schedule, which avoids any unnecessary MSC discarding.

Progressive loss of stem cell functionality caused by the reduction in stem cell number or perturbed cell-cycle activity has been reported in aged animals. Depletion of the stem cell pool with age may occur because these cells lose self-renewal activity and terminally differentiate, thereby exiting the stem cell pool, or because they undergo apoptosis or senescence. Similarly, when MSCs cultured in vitro extensively, reduced self-renewal and accelerated terminal differentiation, as well as enhanced apoptosis or senescence are often observed. Therefore, studying whether extensively passaged aging P10 MSCs still maintain their therapeutic effects after a short-term cell sheet culture on thermo-responsive culture dishes, similar to P3 passaged young MSCs, is important for clinical settings.

Extensive ex vivo culture induces MSC aging: To isolate functional stromal cells it is important not only to study the molecular mechanisms but also for the establishment of stromal cell-based therapeutics. Here the inventor adopted a protocol to isolate human bone marrow derived MSC using a plastic adherent method. Human bone marrow aspirates were obtained from six patients, and plastic-adherent fibroblast-like colonies were observed in all donor samples within the first 5 days of cultivation. Flow cytometry analyses (FIG. 1A) indicated that MSCs obtained from six separate preparations ranged from 84.5% to 97.8% positive for stromal cell markers CD105, CD73 and CD90 in P3 MSCs. However, in P10 MSCs, the positive population of CD105, CD73 and CD90 significantly reduced to 42-13%, suggesting a tendency for loss of stromal phenotype during long-term culture. To eliminate hematopoietic stromal cell contamination, hematopoietic marker CD34 was analyzed. Less than 0.5% of CD34 positive populations in newly isolated human MSCs were observed (data not shown) demonstrating that our MSCs were not contaminated with hematopoietic cells. Finally, the average percentage of positive CD105, CD73 and CD90 MSCs in P3 and P10 cultures from flow cytometry data was quantified and shown in FIG. 1B.

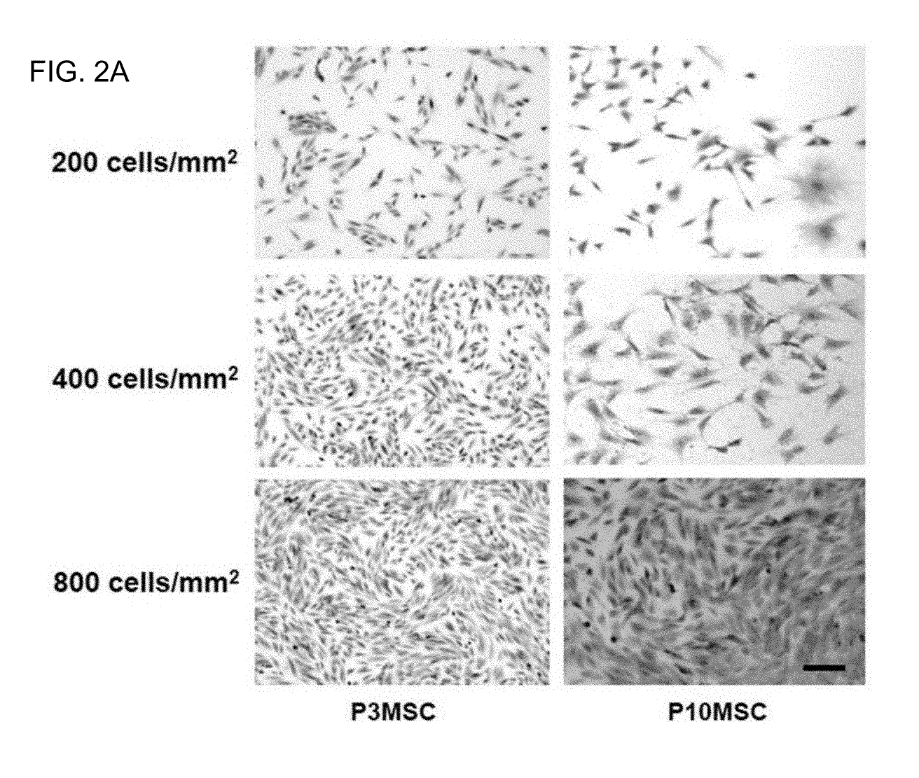

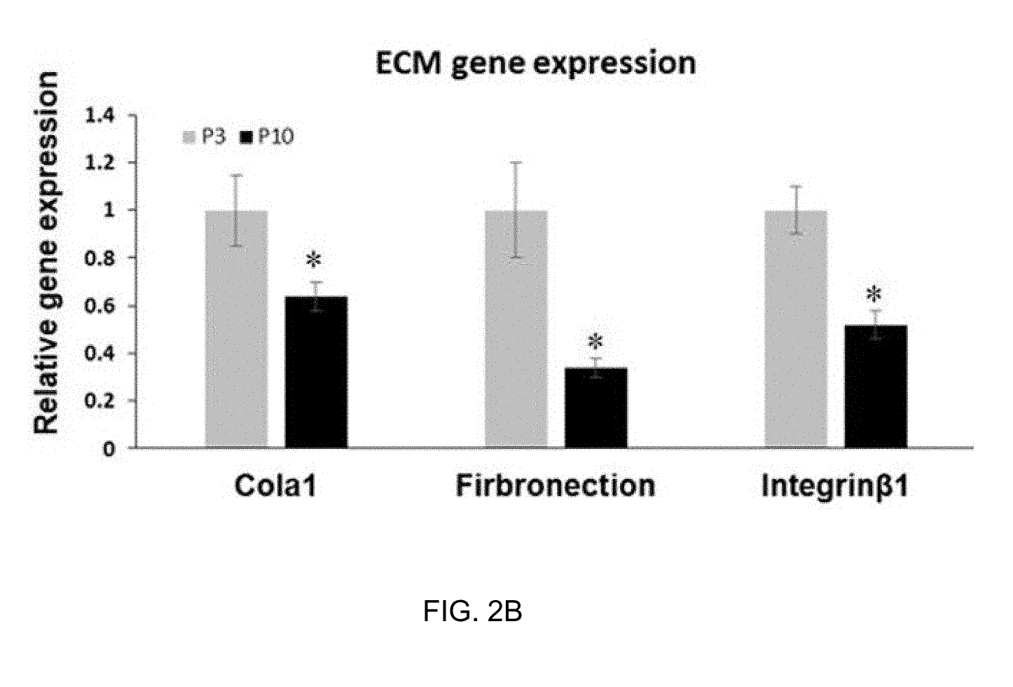

During MSC expansion, the proliferative rates of P3 and P10 cultures were monitored by growth curve and cell doubling time (DT). Initial 20.0×10³ MSCs were seeded to inoculate a 75 cm² culture flask and harvested daily for up to 5 days before reaching confluence. As expected, the cumulative cell number in P3 MSCs was in general significantly higher than that in P10 MSC cultures, indicating a better proliferative activity in P3 MSCs (FIG. 1C). Consistent with this result, the DT mean value in P3 MSCs (26.1±2.5 hours) was much shorter than in P10 MSCs (51.1±4.2 hours), indicating that P3 MSCs required less time to duplicate when compared to P10 MSCs (FIG. 1D). To evaluate the cell morphology change during passaging, MSCs from P3 and P10 were stained by Giemsa solution. At an early phase of P3 culture, more than 90% of MSCs showed a “fibroblast-like” shape and usually formed an almost uniform cell monolayer at confluence (FIG. 2A, left panel). In P10 cultures, the cells varied in size and shape. Some cells became much larger with an irregular and flat shape, and nuclei became more circumscribed in phase contrast microscopy (FIG. 2A, right panel).

To further determine how many MSCs were needed to form a monolayer cell sheet in 24 hours, 3 different densities of P3 and P10 MSCs were seeded into 6-well plates as shown in FIG. 2A. The results clearly showed that a monolayer cell sheet was formed in plates with seeding density of 400 and 800 cells/mm² when P3 MSC was used. Meanwhile, seeding density of 800 cells/mm² was required for P10 MSCs to form cell sheets in 24 hours due to its slower proliferation rate. Cell sheets were then generated for in vivo experiments using seeding density at 400 cells/mm² for P3 MSCs and 800 cells/mm² for P10 MSCs. To further show the difference of ECM in P3 and P10 MSC sheets, the expression of the ECM related genes was measured using total RNA isolated from P3 and P10 MSC sheets, including the type 1 collagen (Cola1), fibronectin, and integrinβ1, that showed a significant decrease of expression in P10 sheets when compared to P3 sheets (FIG. 2B) suggesting a progressive loss of ECM production during long-term culture.

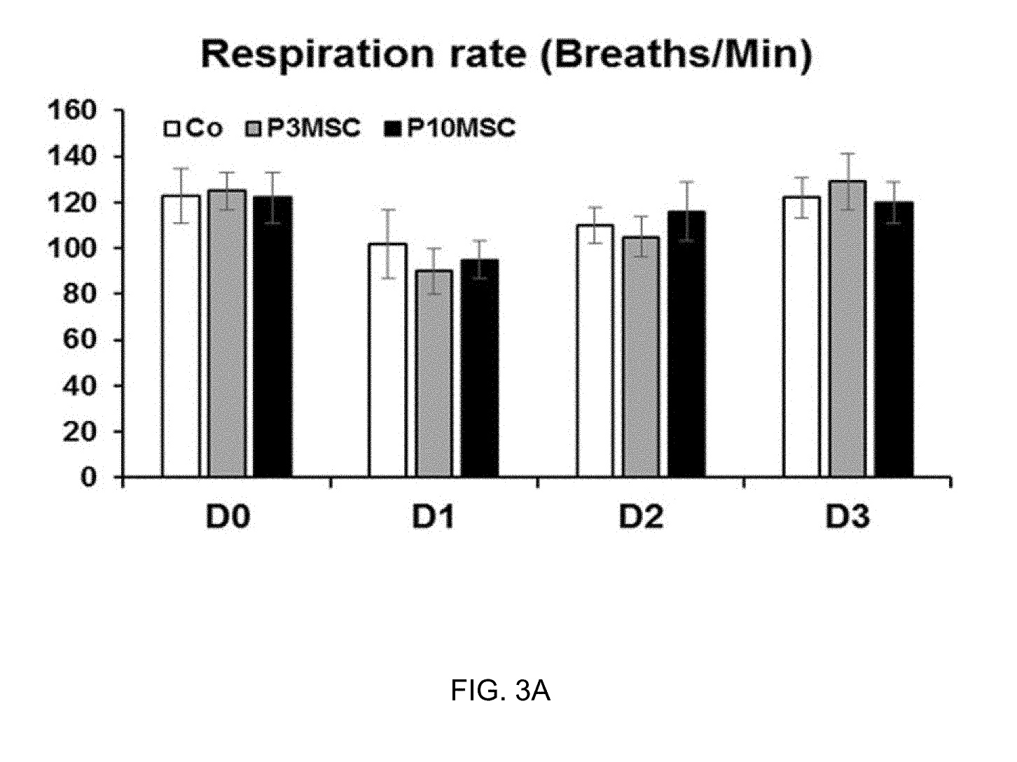

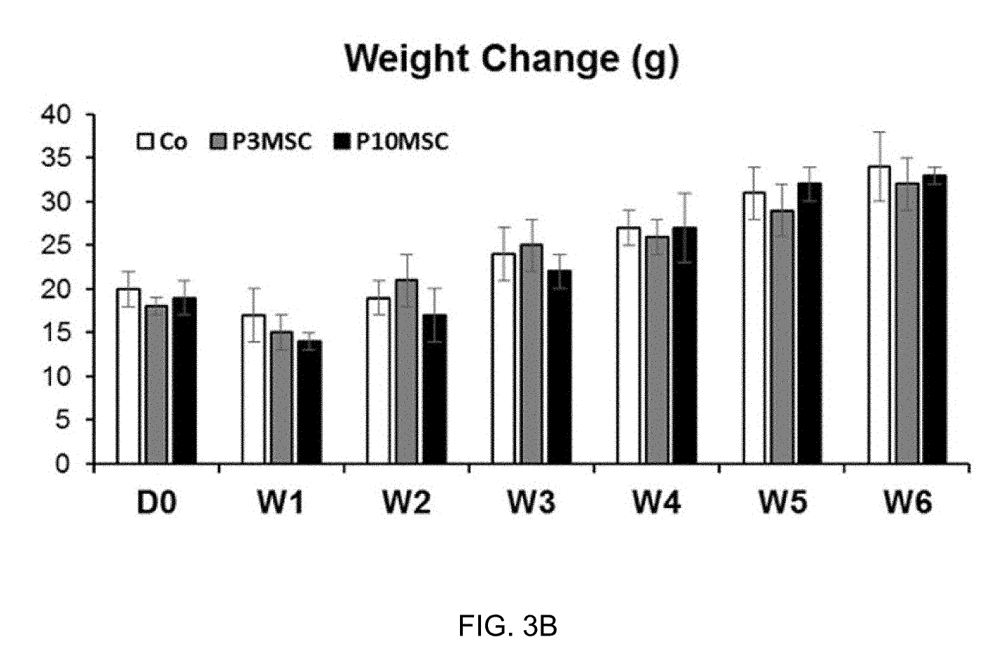

MSC sheets enhance allograft healing: Short-term cell sheet culture does not affect MSC “stemness.” The inventor transplanted human P3 and P10 MSC sheets into a C57/BL6 mouse femur bone allograft model as tissue-engineered periosteum. Observation of the behavior of the animals is most important in the first 3 days of the experiment, since it is expected that rapid or delayed hyperacute transplant rejection may occur. Respiration rates were measured as baseline before allograft surgery (D0) and at 1, 2, and 3 days after surgery. Observation data showed that in all mice, respiration rates measured within the normal range (100 and 200 breaths/min), and no significant difference was noticed between allograft alone and MSC sheet groups (FIG. 3A). Furthermore, among the three groups, no significant changes or differences were observed regarding infection, skin coloration (i.e. redness), posture or activity after surgery. Finally, animals in all three groups showed a slight loss of body weight at one week after surgery, followed by a recovery at 2, 3, 4, 5, and 6 weeks after surgery. The same kinetics within the three groups (FIG. 3B) indicate that human MSCs transplantation is well tolerated in this allograft mouse model.

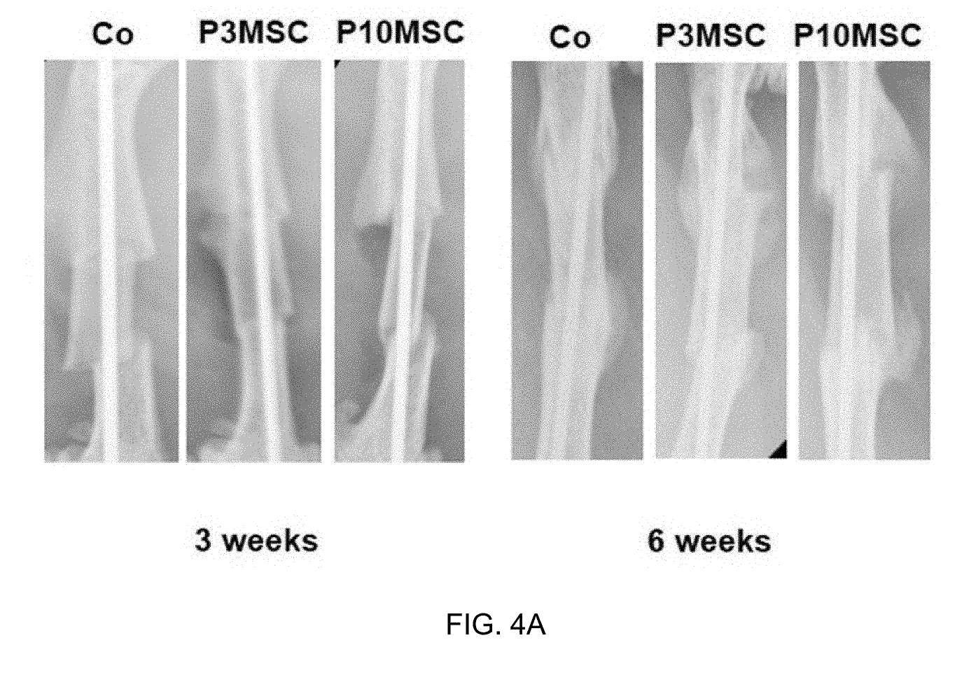

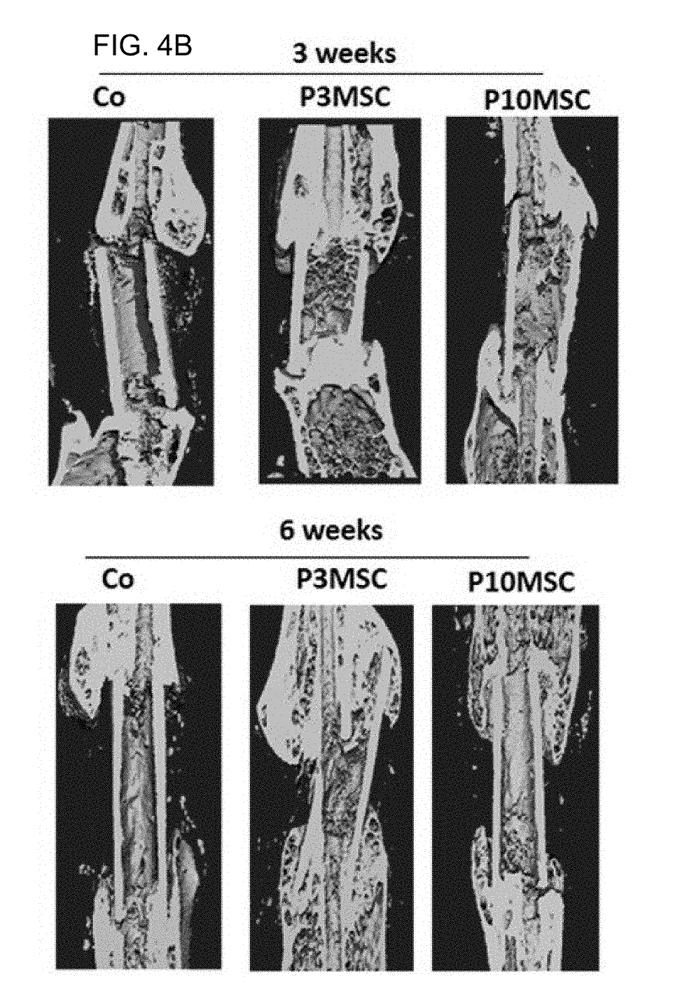

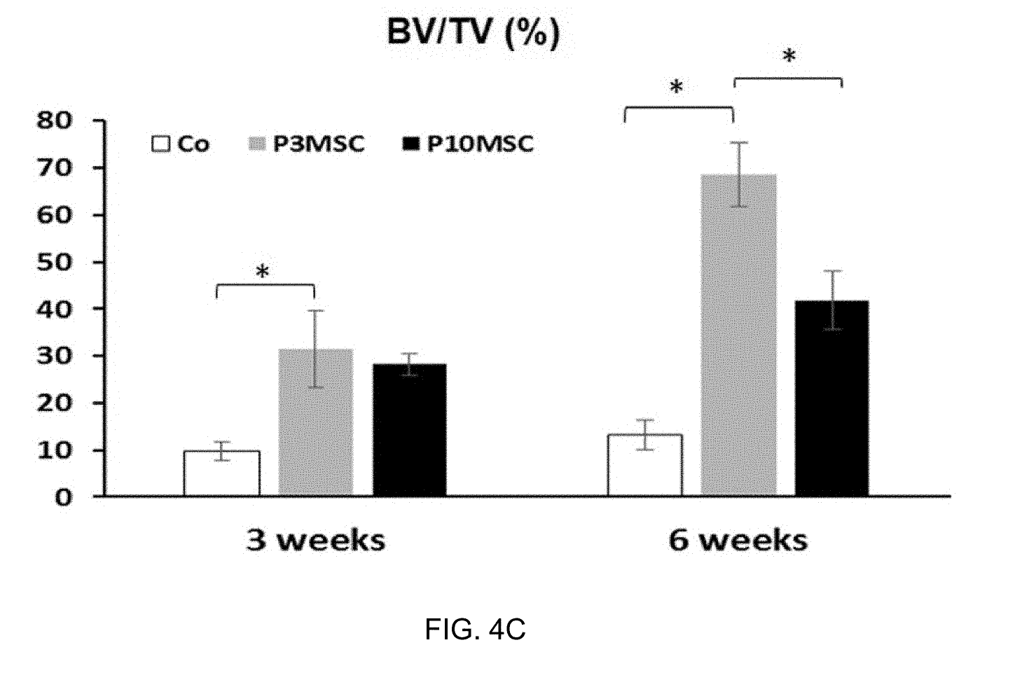

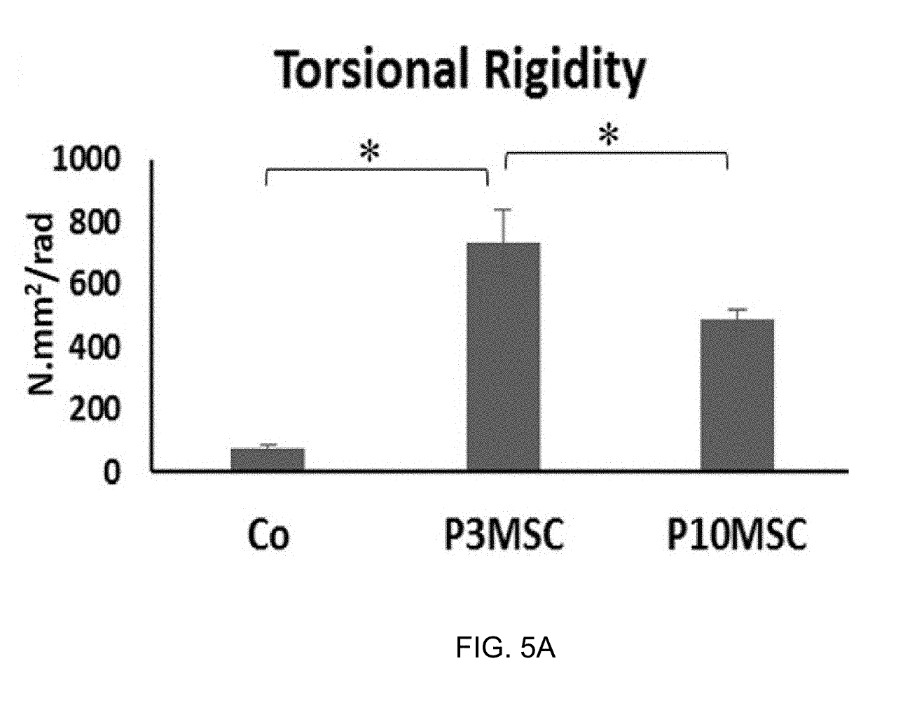

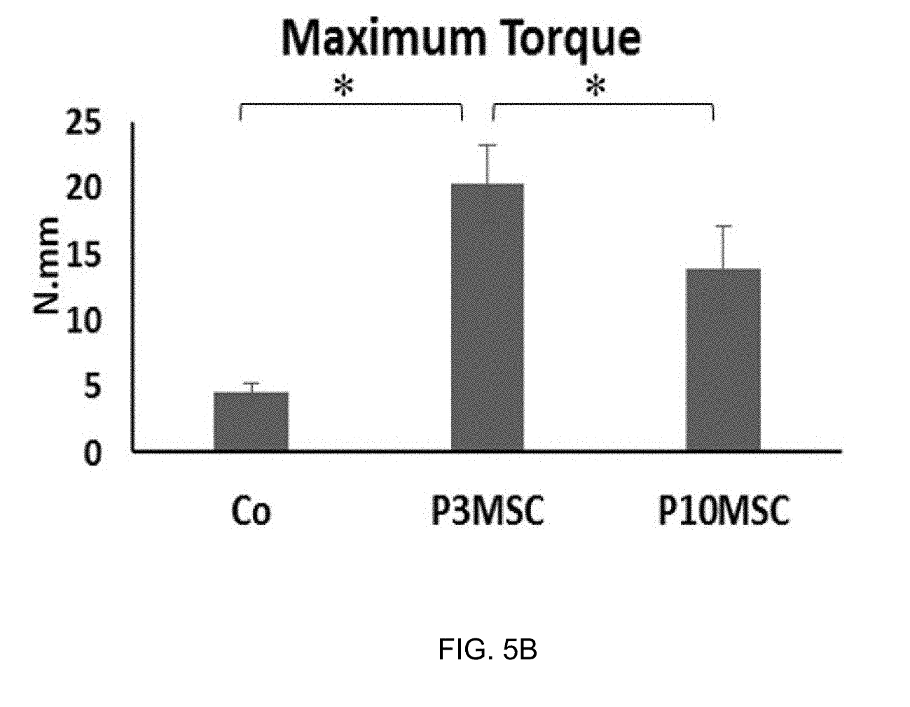

To analyze the allograft healing process, new bone callus formation was monitored using X-ray (FIG. 4A). At 3 weeks post-surgery, the fracture line between the host bone and allograft disappeared in both sheet-wrapped groups. However, in the control graft alone group, the fracture line remained at this time point, indicating more bony callus formed between allograft and host bone ends in both P3 and P10 MSC sheets wrapped allograft groups. At 6 weeks post-surgery, although a greater number of bony callus were observed in both MSC sheets groups when compared to allograft alone groups, the bony callus area in P10 MSC sheet groups was significantly less than that in P3 MSC sheet groups. To further validate the X-ray findings, Micro-CT analysis was performed to quantify the newly formed bony callus at 3 and 6 weeks post-surgery (FIG. 4B). Consistent with X-ray findings, the CT results clearly showed that the size of the newly formed bony callus in P3 MSC sheet groups was larger than that in the P10 MSC sheet and allograft alone groups. Although the percentage of BV to TV in P10 MSC sheet groups was less than in P3 MSC sheet groups, P10 MSC groups indeed showed a better callus formation when compared with allograft alone groups (FIG. 4C). Since the success of bone repair should be defined by restoration of the biomechanical properties of the original bone, we next performed torsional testing to measure the strength of the bone allograft samples at 6 weeks post-surgery. As would be expected from the known volume of bony callus in Micro-CT data, both P3 and P10 MSC sheet groups had significantly higher torsional properties than did the allograft alone groups. Torsional rigidity for P10 MSC-sheet groups (486.3±68.1 N·mm²/rad) was 10-fold higher than in allograft alone groups (49.3±7.4 N·mm²/rad), whereas the torsional stiffness for the P3 MSC sheet groups (735.6±118.9 N·mm²/rad) was 15 times greater (FIG. 5A) than in allograft alone groups. Maximal torque analysis further showed that P3 MSC sheet groups had the highest value of peak torque in the three different allograft groups, confirming the best stability and integration with the native bone (FIG. 5B).

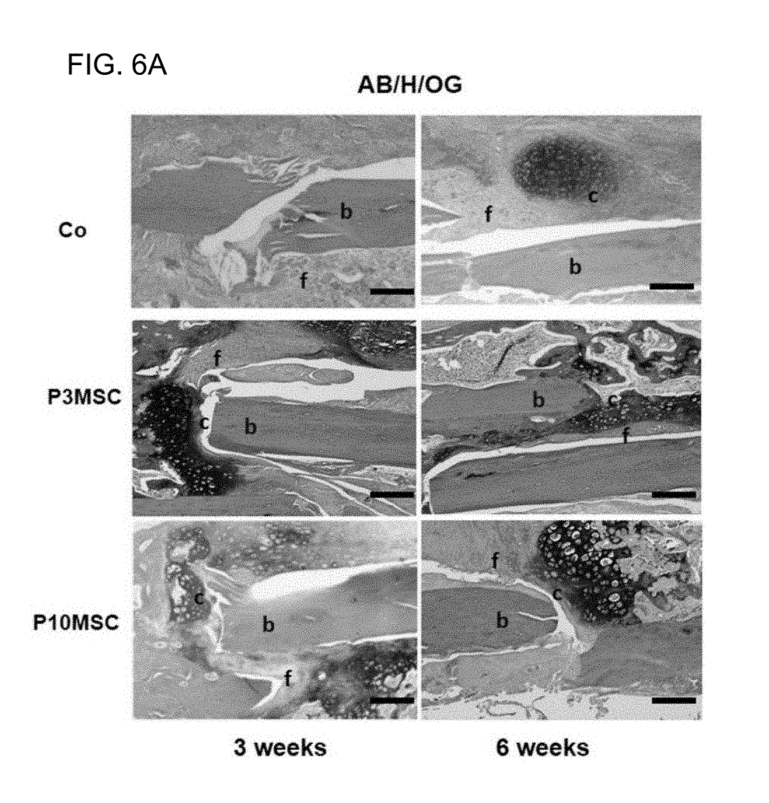

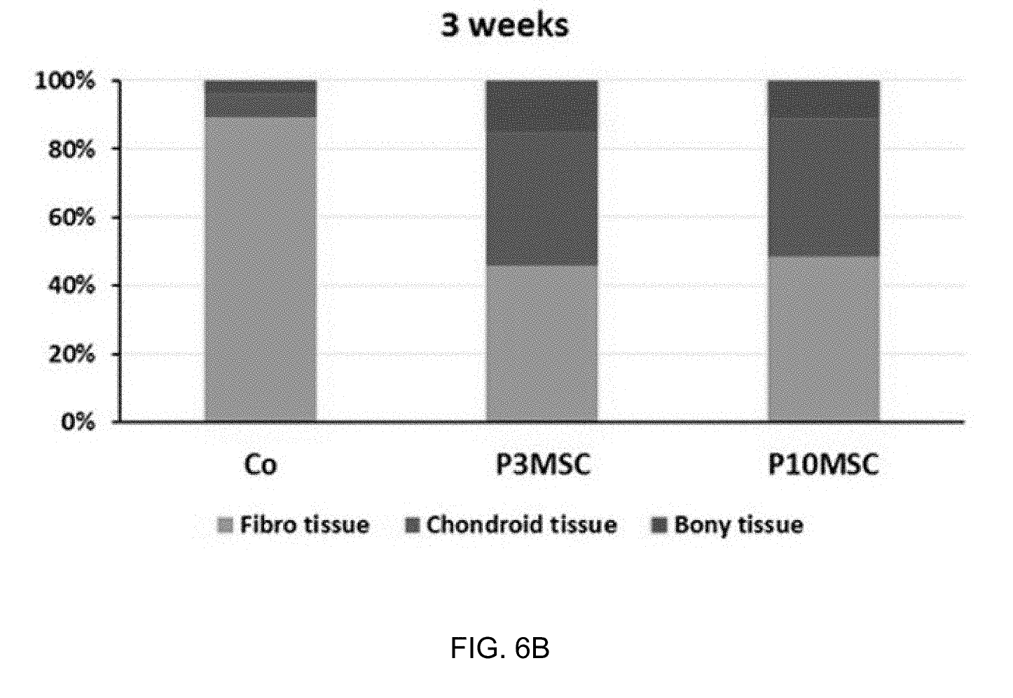

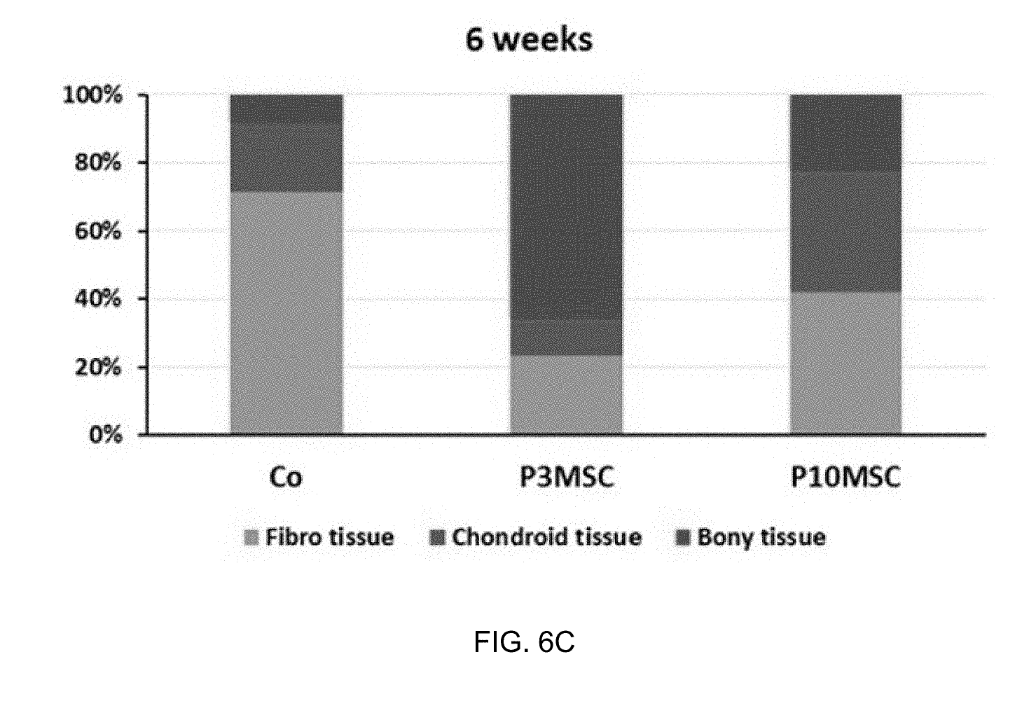

Delayed bony callus calcification in aged MSC sheet-wrapped allograft healing: To study the histologic changes in callus, AB/H/OG staining was performed in samples from 3 and 6 weeks post-surgery. Sections from 3 weeks after surgery showed that larger chondroid tissues and hard callus were formed surrounding the gaps between allograft and host bone in both MSC sheet-wrapped allograft groups (FIG. 6A, left) when compared with control allograft alone groups suggesting an enhanced chondro-osteogenesis in both cell sheet groups. At 6 weeks post-surgery, abundant chondroid tissues filling the site between host bone and allograft were still observed in the P10 MSC sheet group, while the size of the chondroid tissues was significantly smaller in the P3 MSC sheet groups. In contrast, the bony callus area in P3 MSC sheet groups was much larger than in P10 MSC sheet groups, indicating an accelerated endochondral ossification in P3 MSC sheet groups (FIG. 6A, right). Histomorphometric analysis of histologic sections further confirmed that although both MSC sheet groups had a similar amount of chondro-osteoid callus at 3 weeks post-surgery (FIG. 6B), more rapid bony callus was formed in P3 MSC sheet groups when compared with P10 MSC sheet groups at 6 weeks post-surgery (FIG. 6C).

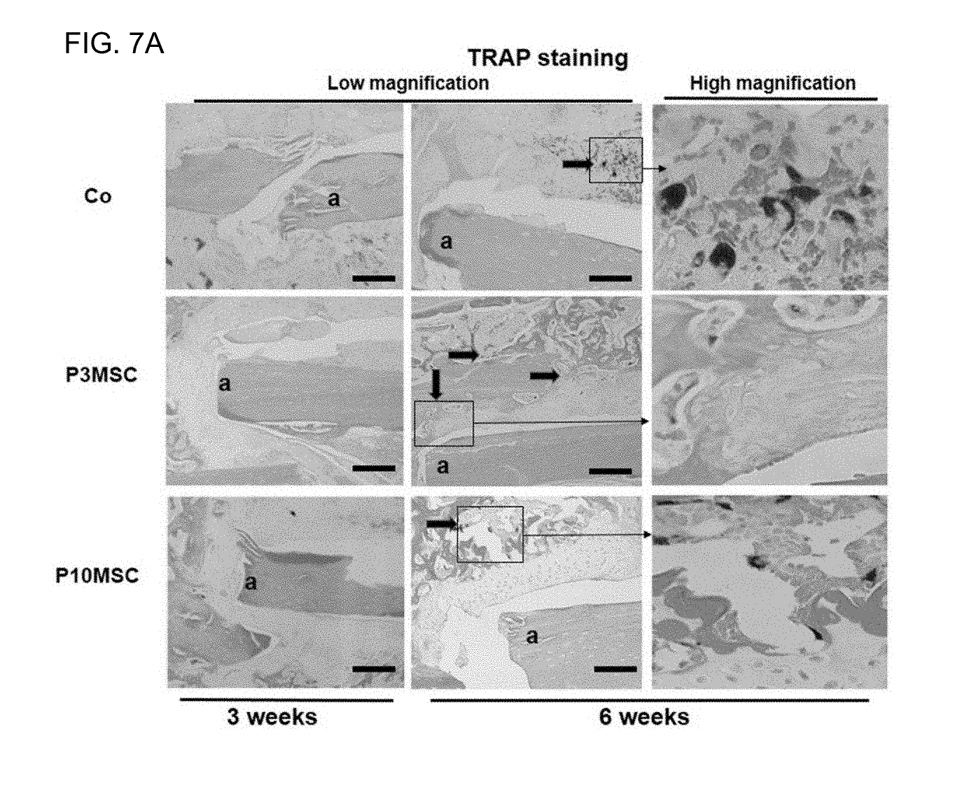

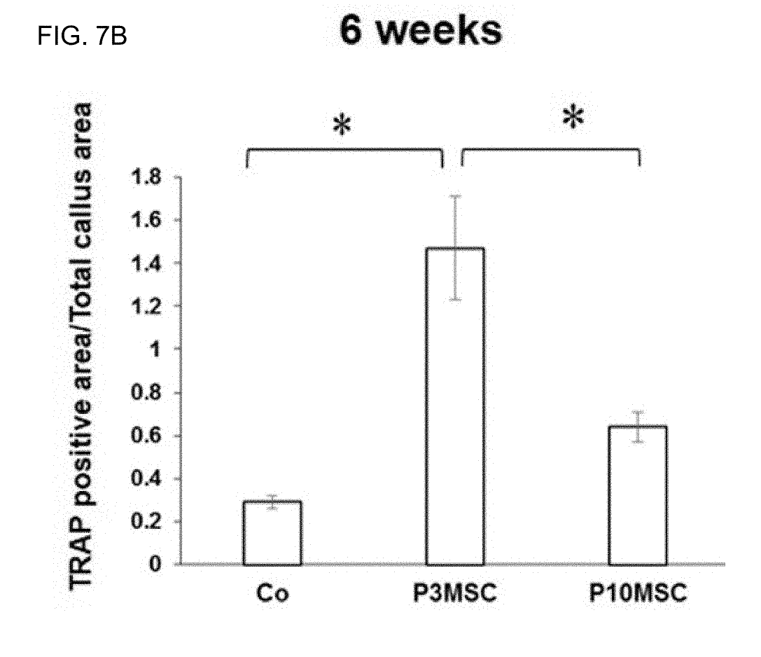

Enhanced osteoclast activity in P3 MSC sheet-mediated bony callus formation: Since the ossification of fibrocartilaginous callus requires organized osteoblast and osteoclast activity, TRAP staining was performed to determine whether osteoclast is involved in this callus mineralization process. TRAP staining results from sections at 3 weeks did not show significant staining or difference in newly formed callus among the three different groups (FIG. 7A, left). In contrast, TRAP staining showed an increased chondro-osteoclast activity in callus at 6 weeks post-cell sheet transplantation (FIG. 7A, right). Particularly, there were limited chondro-osteoclasts observed at small area in control callus, while more chondro-osteoclasts were widely distributed in large area of callus in P3 MSC sheet groups. When quantification analysis was performed with TRAP staining, significantly more osteoclast activity was observed in P3 MSC sheet groups compared to P10 MSC and allograft alone groups (FIG. 7B).

Discussion: It is estimated that approximately 1.6 million bone grafts are used each year to regenerate bone that was lost due to trauma or disease. Today, the “gold standard” of bone grafts used in technology is still an autograft. Unfortunately, the harvest of an autograft is not always possible and might lead to co-site morbidity and excessive pain. Another option is the bone allograft, which is readily available for transplantation. These bone allografts have been preserved from deceased persons who have been carefully screened for diseases, including HIV and hepatitis infection. The host bones will first grow into the allograft, then replace the allograft with the host bone. Rejection of the grafted bone is rare because there are few fragments of the donor's cells in the allograft to induce an immunoreaction. The greatest problems with allograft reconstruction are the chance of graft fracture; and the failure of healing between the graft and the adjacent patient bone due to limited bone formation capability. Therefore, there is a clear clinical need to develop revitalized allografts that are comparable or close to autografts to efficiently replace the missing bone tissue. To address this major clinical problem, the inventor has developed a “tissue-engineered periosteum” composed of an MSC sheet that has been extensively tested in animal models. The inventor has demonstrated that MSC sheets generated with fresh isolated cells possess enhanced potential for both chondrogenic and osteogenic differentiation. There are clear-cut biologic advantages of MSC sheets for bone regeneration.

To generate high-quality MSCs for research and future clinical application, human bone marrow MSC was first isolated from 6 patients using a modified cell plastic adherent method. In accordance with the literature, immunophenotypic analysis of all MSC preparations confirmed that the detection level for positive markers was much higher in early passages at 3 compared to late passages at 10. Furthermore, some P10 MSCs significantly changed their initial morphology, and the cytoplasm began to be granular with increased cell size and reduced capability to reach a confluent monolayer suggesting an aging process in cells. Most of the P3 MSCs maintained their initial morphology, continuing to generate a very dense cell monolayer. These results support the notion that long-term culture has an incremental impact on “stemness” potential of MSCs.

Population doubling times were also recorded to calculate the time that cells took for doubling their number. As expected, P10 MSCs took much longer time for doubling than P3 MSCs. Since the proliferation rate of P10 MSC is low, the cell number needed for generating a cell sheet in 24 hours was measured. The results clearly showed a double amount of aged P10 MSCs is needed for generation of monolayer cell sheets when compared to P3 MSCs.

To determine whether aged MSCs could be used for bone tissue regeneration, both young P3 and aged P10 MSC sheets were transplanted into a femoral allograft mouse model and were compared to allograft alone without MSC sheets. MSCs have negligible immunogenicity and the capacity for immune suppression. MSCs express low levels of major histocompatibility complex (MHC) class I molecules but lack expression of MHC class II molecules and the co-stimulary molecules CD80, CD86, and CD40. In addition, MSCs inhibit dendritic cells, T cells, B cells natural killer cells, and macrophages. Based on these facts, wild type mouse was selected to use for in vivo MSC transplantation. Since MSCs are immunoprivileged, gross observations were performed to confirm the overall safety of human MSC sheet transplantation. As respiratory rate is an acute toxicity parameter for excessive inflammatory cytokines (such as tumor necrosis factor alpha [TNFα], interleukin [IL]-1α and IL-1β) aggregate in the microvasculature of the lungs that may cause cardiac and pulmonary distress, mouse respiratory rate was monitored before and after surgery. There were no significant differences between allograft alone and MSC sheet groups regarding evaluation of respiratory distress upon human MSC transplantation. Gross observation analysis further showed that no significant difference was noticed in the three groups regarding the animal activity, skin color change, or weight loss, demonstrating the safety of transplantation of human MSCs into a mouse.

To further determine the therapeutic effect of aged P10 MSC sheets for bone tissue regeneration, a pre-clinic mouse model with a critical-sized bone defect was used in this study. Our data showed an elevated early soft callus formation in P10 MSC groups that is comparable to P3 MSCs at 3 weeks after surgery when compared to allograft alone groups. However, significantly less bony callus was observed surrounding P10 MSC sheet-wrapped allografts when compared with P3 MSC groups at 6 weeks post-surgery, indicating a delayed bony callus formation in P10 MSC sheet groups. Chondroid tissue ossification is a dynamic process marked by chondro-osteoclast mediated hypertrophic chondrocytes removal, type II collagen degradation and osteoblast-mediated type I collagen formation. To further look at the possible mechanism responsible for the difference between P10 and P3 MSC sheet-mediated soft callus mineralization, the chondro-osteoclast positive area was quantified in the newly formed callus by TRAP staining. The data clearly showed that osteoclast activity in the P3 MSC group was significantly higher than in P10 MSC groups at 6 weeks post-surgery, but not at 3 weeks. This increased chondro-osteoclast activity leads to accelerated removal of chondriod callus in P3 MSC group. In contrast, less numbers of chodro-osteoclasts in P10 MSC groups delayed the chondriod callus resorption and subsequent callus remodeling by showing more significantly chondriod callus formed in the aged MSC sheet groups. These data indicate the accelerated callus calcification and remodeling in P3 MSCs may be largely due to more osteoclasts in newly formed callus.

In summary, this study provides some insights on the differences between early-passaged young P3 MSC sheets and later-passaged aged P10 MSC sheets regarding the biological properties and chondro-osteogenic ability. Compared to young MSC sheets, aged MSC sheets show an equal chondrogenic effect and a reduced callus calcification.

Materials and Methods

Ethics Statement and Donor Data: Human bone marrow aspirate was harvested from the iliac crest of donors (n=6), who were patients at University of Rochester Medical Center, had spine fusion surgery using their own bone marrow aspirate for MSC isolation. The mean age of the donors was 55 years (range 45-70). Donors included four males and two females with no history or evidence of genetic disease or malignancy. The Ethics Committee of University of Rochester approved the use of patient's tissue material for this study (RSRB00048032). Written informed consent was obtained from all donors, and no identifying information will be recorded in this study. All methods for human experiments were performed in accordance with relevant guidelines and regulations.

Human MSC isolation and culture: The method for isolation of human MSC was based on red blood cell (RBC) lysis with ammonium chloride. Briefly, 8 ml bone marrow aspirates from each patient were combined with 0.5 ml heparin anticoagulant (Becton Dickinson, San Jose, Calif., USA; BD) in a syringe and delivered to our lab. After incubation in RBC lysis buffer for 10 min, total bone marrow cells were seeded at 5.0×10⁶ cells/cm² in alpha-minimum essential medium (α-MEM) supplemented with 15% FBS, 2 mM L-glutamine, 0.5% antibiotic/antimycotic solution (all from Gibco-BRL, Life Technologies) for 3 days. Newly formed cell colonies were washed 3-5 times with 1×PBS and incubated at 37° C. in a humidified 95% air and 5% CO₂ atmosphere, cultured up to 80% confluence, and then trypsinized (trypsin-EDTA solution, Gibco-BRL), centrifuged, and re-plated at a density of 20,000 cells/cm² as P1 MSCs for subsequent expansion. After expansion for 2 passages, some of the P3 cells were harvested for flow cytometry analysis. The remaining P3 cells were divided into 2 populations for cell sheet formation as P3 MSCs and for continued passage until harvested as P10 MSCs for cell sheet culture. The MSCs from the patient with the highest expression of stromal markers were used to generate cell sheets in this study, and long-term cultures were always passaged at the same density of about 70-80% confluence. In vitro cell growth was monitored by cell number count and cumulative population doubling time (DT) calculation. DT was examined using the formula: (t−t0)·log 2/log(N−N0), where t−t0 is culture time in hours, N is the number of harvested cells, and N0 is the number of cells in the initial seeding.

Flow cytometry analysis: MSC phenotype was evaluated using P3 and P10 MSCs by the expression of CD34-APC (BD Biosciences), CD90-PE (BD Biosciences), CD73-PE (BD Biosciences) and CD105-FITC (R&D Systems, Minneapolis, Minn.). As previously described, cells were incubated for 30 min at room temperature with antibodies, and flow cytometry was performed on an LSR-II flow cytometer (Beckton Dickson). Isotype matched controls were used to set the electronic gates on the flow cytometer. The data were analyzed using FlowJo software (Tree Star).

Mouse study design: Animal use in this study was approved by Animal care and use committee at Louisiana State University Health Sciences Center. Allogeneic bone grafts were obtained from mice of the 129/J strain for implantation into C57BL/6 J mice. The Louisiana State University Committee of Animal care and use approved all animal surgery procedures (protocol#: P-15-005). Experiments were designed to include 12 male mice per group at different time points. Host mice carrying allografts were randomly and equally assigned to 3 groups: control (allograft alone), P3 MSC sheet/allograft and P10 MSC sheet/allograft groups. The sample size (n=6) for Micro-CT and biomechanical testing was determined by power analysis based on our previous experiment data. All methods for animal experiments were performed in accordance with relevant guidelines and regulations.

Generation of MSC sheets: MSCs from P3 and P10 cultures were re-seeded at 200, 400, and 800 cells/mm² on thermo-responsive 6-well culture plates with UpCell surface (Thermo Scientific, Cat. 174901) for cell sheet formation. After 24-hour culture, the cell sheet culture was monitored by Giemsa staining as previously reported. The selected cell seeding density was used to generate a monolayer cell sheet (100% confluence) in 24 hours for subsequent in vivo implantation. Total RNA was extracted from both P3 and P10 MSC sheet cultures, Real time-PCR was performed as we previously described for ECM related genes: fibronectin, integrin β1, and type 1 collagen.

Devitalization of bone allografts: Eight-week-old male 129/J mice were used for donation of devitalized allografts. Mice were euthanized, and a 4 mm mid-diaphyseal segment was removed from each femur by osteotomy using a rotary Dremel with custom circular diamond blades. Allograft segments were flushed of the bone marrow using 25-gauge needles, the periosteal tissues were manually stripped, and the bone grafts were washed repeatedly in 70% ethanol for at least 4 hours. The allografts were then stored in 100% ethanol at −80° C. for at least 7 days to complete the devitalization process.

Wrapping of MSC sheets on allografts: Following MSC sheet formation, cell sheets were wrapped onto devitalized allografts. The cultured MSC sheets were covered by a cell transfer membrane (Thermo Scientific, Cat. 1749016) and kept at 25° C. for 10 minutes. After the cell layer adhered to the membrane, it was detached carefully from the thermo-responsive culture plate. The cell sheet and membrane were then placed in a new, larger dish with the cell layer facing up; next, the devitalized allografts were placed on top of the cell sheets, wrapped in one layer of cell sheet and incubated for an additional 30 minutes at 37° C. in fresh media to release the membrane. After carefully removing the membrane from the cell sheet, the MSC sheet wrapped allografts were kept at 37° C. for allograft transplantation surgery.

Surgical reconstruction of the mouse femoral defects: Eight-week-old C57BL/6J mice were anesthetized via intraperitoneal injection with a combination of ketamine (60 mg/kg body weight) and xylazine (4 mg/kg body weight). A 7-8 mm incision was made, and the midshaft femur was exposed using blunt dissection of muscles. A 4-mm mid-diaphyseal segment was removed from the femur by osteotomizing the bone using the Dremel tool. A 4-mm bone graft with/without MSC sheets was then inserted into the segmental defect and stabilized using a 26-gauge metal pin placed through the intramedullary marrow cavity. The incisions were closed using silk sutures. Graft healing was followed radiographically using a Faxitron X-ray system (Faxitron X-ray Corporation, Wheeling, Ill.). Mice were sacrificed at 3 and 6 weeks post-surgery, and samples processed for further analysis.

Safety evaluation: Safety parameters were evaluated, including respiration rates, activity, posture and skin color change caused by inflammation in the suture area before and 3 days after surgery. Weight loss was also monitored once a week for up to 6 weeks.

Micro-CT bone imaging analyses: Some of the reconstructed femurs from week 3 and week 6 (n=6) were imaged after careful dissection and removal of the intramedullary pin using a Micro-CT system (VivaCT 40, Scanco Medical). The femurs were scanned using a high-resolution (10.5 microns) x-ray with energy settings of 55 kVp and 145 μA. Quantification of bone volume (BV) and total volume (TV) of callus was performed as previously described using the Scanco analysis software.

Histological evaluation of grafted femurs: The femoral samples (n=6) to be used for Micro-CT analyses were then fixed in neutral buffered formalin and processed in paraffin. Paraffin-embedded samples were sectioned at 5 μm and stained with Alcian Blue/Hematoxylin/Orange-G (AB/H/OG) to determine the contributions of cartilage, bone, and fibrotic tissue during the repair process using OsteoMeasure™ software. Tartrate-resistant acid phosphatase (TRAP) staining was further performed in paraffin sections using kit (387 A) from sigma-aldrich. Positive area was quantified by ImageJ software.

Biomechanical testing: Specimens (n=6) from 6 weeks were harvested and moistened with saline before biomechanical testing. The ends of the femurs were cemented into square aluminum tube holders using polymethylmethacrylate (PMMA) to ensure axial alignment and to maintain a gage length of 7-8 mm, allowing a length of at least 3 mm to be potted at each end. Specimens were mounted on an EnduraTec TestBench™ system (200 N·mm torque cell; Bose Corporation) and tested in torsion until failure. The torque data were plotted against the rotational deformation (normalized by the gage length and expressed as rad/mm) to determine the maximal torque and torsional rigidity as we reported previously.

Statistical analysis: The above experiments were repeated at least three times independently. All data are presented as mean±standard deviation (SD). Statistical significance among the groups was assessed using one-way analysis of variance (ANOVA). The level of significance was P<0.05.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A tissue engineered periosteum for a patient comprising:
a sheet of stem cells.

2. The tissue engineered periosteum of claim 1 wherein the stem cells are mesenchymal stromal cells (MSC).

3. The tissue engineered periosteum of claim 1 wherein the MSC are isolated from a bone marrow of the patient.

4. The tissue engineered periosteum of claim 1 wherein the passage number for the MSC is 10, 9, 8, 7, 6, 5, 4, 3, 2 or 1.

5. The tissue engineered periosteum of claim 1 wherein the passage number for the MSC is 3 or less.

6. The tissue engineered periosteum of claim 1 wherein the passage number for the MSC is 3.

7. A method of repairing bone injury on a patient comprising
implanting into the patient an engineered bone graph comprising a scaffold wrapped in a tissue engineered periosteum.

8. The method of claim 7 wherein the tissue engineered periosteum includes a sheet of stem cells.

9. The method of claim 7 wherein the tissue engineered periosteum includes a sheet mesenchymal stromal cells (MSC).

10. The method of claim 7 wherein the scaffold is formed of one of an allograph, an autograph, and of one of one or more metals, one or more synthetic polymers, and a combination of one or more metals and one or more synthetic polymers.

11. The method of claim 7 wherein the bone injury is one of a traumatic extremity injury, a skeletal tumor resection, and a large bone defect.

12. A tissue engineered bone graph comprising:
a scaffold;
a tissue engineered periosteum wrapped around an exterior of the scaffold.

13. The tissue engineered bone graph of claim 12 wherein the scaffold is an allograph.

14. The tissue engineered bone graph of claim 12 wherein the scaffold is an autograph.

15. The tissue engineered bone graph of claim 12 wherein the scaffold is formed of one of one or more metals, one or more synthetic polymers, and a combination of one or more metals and one or more synthetic polymers.

16. The tissue engineered bone graph of claim 12 wherein the tissue engineered periosteum is a sheet of low passage mesenchymal stromal cells (MSC).

17. The tissue engineered bone graph of claim 16 wherein the passage number for the MSC is three or two.

18. The tissue engineered bone graph of claim 12 wherein the tissue engineered periosteum is wrapped around an area adjacent to a terminal end of the scaffold.
